(19) United States
(12) Patent Application Publication
(10) Pub. No.: US 2021/0077363 A1
(43) Pub. Date: Mar. 18, 2021

(54) SPRAYABLE COSMETIC COMPOSITION HAVING A COLOR EFFECT

(71) Applicant: Henkel AG & Co. KGaA, Duesseldorf (DE)

(72) Inventors: DIANE METTEN, Hamburg (DE); ROLF BAYERSDOERFER, Hamburg (DE); THORSTEN KNAPPE, Schenefeld (DE)

(73) Assignee: Henkel AG & Co. KGaA, Duesseldorf (DE)

(21) Appl. No.: 16/642,752

(22) PCT Filed: Aug. 27, 2018
(86) PCT No.: PCT/EP2018/072964

§ 371 Date: Feb. 27, 2020

(30) Foreign Application Priority Data
Nov. 21, 2017 (DE) 10 2017 220 773.6

(51) Int. Cl. A61K 8/02; A61Q 5/06; A61K 8/04; A61K 8/34; A61K 8/73

(52) U.S. Cl. CPC A61K 8/027; A61Q 5/065; A61K 8/046; A61K 2800/43; A61K 8/732; A61K 2800/412; A61K 8/34

(57) ABSTRACT

Cosmetic compositions for the cosmetic treatment of keratinous fibers, containing, in each case based on their weight:
(a) fibers,
(b) ethanol,
(c) at least one polymer,
(d) propellant,
(e) from 0 to about 10% by weight of dye(s),
(f) from 0 to about 10% by weight of pigment(s),
   with the proviso that the sum of the amounts of ingredients (e) and (f) is from about 0.0001 to about 10% by weight, to increase the hair volume and cause an increase in the fullness and the feeling of “having more hair”. The coloring of the products also allows coverage of bald areas without causing abrasion on hands or clothing or pillows.

### CROSS-REFERENCE TO RELATED APPLICATION

This application is a U.S. National-Stage entry under 35 U.S.C. § 371 based on International Application No. PCT/EP2018/072964, filed Aug. 27, 2018, which was published under PCT Article 21(2) and which claims priority to German Application No. 10 2017 220 773.6, filed Nov. 21, 2017, which are all hereby incorporated in their entirety by reference.

### TECHNICAL FIELD

The present application concerns sprayable cosmetic compositions for treating keratinous fibers, containing, in addition to a solvent and a propellant, fibers and at least one colorant. These compositions are suitable for a wide range of cosmetic uses, but in particular for the temporary shaping of keratinous fibers while simultaneously coloring the fibers. The application also concerns the cosmetic use of these products and hair cosmetic processes using them.

### BACKGROUND

The present disclosure was based on the task of providing products which increase the hair volume and achieve an increase in fullness and the feeling of “having more hair”. By coloring the products, it should at the same time be possible to cover bald spots without causing abrasion on the hands or clothing or pillows.

The older German patent application DE 10 2017 205 558.8 concerns a sprayable cosmetic composition for the treatment of keratinous fibers which, in addition to a solvent and a propellant, also contains fibers. These compositions are suitable for various cosmetic purposes, but especially for the temporary shaping of keratinous fibers. Furthermore, this older German patent application relates to the cosmetic use of these products and hair cosmetic processes using these products.

It has now been found that the corresponding compositions may be dyed and/or mixed with dyes or pigments and are then excellently suited for a number of other cosmetic purposes and for solving the task as contemplated herein.

### BRIEF SUMMARY

Cosmetic compositions for the treatment of keratinous fibers are provided. In an exemplary embodiment, a cosmetic composition includes (a) fibers, (b) ethanol, (c) at least one polymer, (d) a propellant, and one or more of (e) a dye and/or (f) a pigment. A sum of the quantities of the dye (e) and the pigment (f) is from about 0.0001 to about 10 weight percent, based on the total weight of the cosmetic composition.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The subject-matter of the present disclosure is a cosmetic composition for the cosmetic treatment of keratinous fibers, containing, in each case based on its weight:
(a) fibers
(b) ethanol
(c) at least one polymer
(d) propellant
(e) from 0 to about 10% by weight of dye(s)
(f) from 0 to about 10% by weight of pigment(s)
   provided that the sum of the quantities of ingredients (e) and (f) is from about 0.0001 to about 10% by weight.

The cosmetic composition contains fibers as its first essential component. The addition of these fibers influences the application properties of the sprayable hair cosmetic in an unexpected way. The spray pattern and the application of the sprayed cosmetic become more even. In a standardized test arrangement, the addition of the fibers at the same pressure in the spraying device and at the same distance from the area to be sprayed also causes a surprising increase in the sprayed area achieved.

Stretched, flexible structures made of a fiber material with a length to diameter ratio above about 3:1, preferably above about 5:1 and in particular above about 10:1 are used as the fibers. Fibers cannot absorb compressive but only tensile forces in the longitudinal direction.

The length of preferably used fibers is in the range of from about 1.0 to about 200 μm, preferably from about 2.0 to about 150 μm and especially from about 5.0 to about 100 μm. Of course, fibers of greater fiber length, for example fibers with a length above about 200 μm or above about 300 μm, may also be used.

The fiber material may be of natural or synthetic origin. Natural fibers are all those fibers which are obtained from plant, animal or mineral material without chemical modification.

Plant fibers are found in plants as vascular tissue in the stalk, in the stem, in the bark or as seed extensions. As a rule, plant fibers are mainly composed of cellulose. Corresponding fibers are summarized under the term cellulose fibers.

In a preferred embodiment, the fibers used in cosmetic compositions are selected from the group of fibers made of natural fiber materials. The use of cellulose fibers is particularly preferred, especially cotton fibers, flax fibers and kapok fibers. Furthermore, the use of animal fibers from the group of silk fibers, cashmere fibers and wool fibers is preferred. The use of cellulose fibers is particularly preferred.

Alternatively or in combination with the natural fibers described above, fibers based on synthetic fiber materials may also be used in the cosmetic compositions.

The use of synthetic fiber materials obtained by modifying plant or animal starting materials is particularly preferred. The group of these preferred fibers includes fibers made of viscose, polylactic acid, alginate, chitin or chitosan.

Other preferred synthetic fiber materials are polyamides, polyesters and polyhydroxyalkanoates.

Fiber weight percentages of from about 0.1 to about 5.0%, preferably from about 0.1 to about 2.0% by weight, especially preferably from about 0.1 to about 1.0% by weight and especially from about 0.1 to about 0.5% by weight of the total weight of the cosmetic composition have proven to be particularly beneficial for the technical and cosmetic effect.

The cosmetic compositions contain ethanol as their second essential ingredient. The percentage by weight of ethanol in the total weight of the cosmetic composition is preferably from about 45 to about 70%, preferably from about 40 to about 65% and more particularly from about 50 to about 60%. The fibers are suspended in ethanol.

The third essential ingredient of the cosmetic composition is the polymer. For the applicability and cosmetic effect of the compositions, it has been shown to be advantageous to limit the percentage by weight of the polymer in the total weight of the composition to from about 0.5 to about 8.0%, preferably from about 1.0 to about 6.0% and in particular from about 2.0 to about 4.0%.

Polymers are used in cosmetic compositions, for example, because of their firming and/or film-forming properties. Due to their cosmetic effect, the use of film-forming polymers is particularly preferred.

Suitable polymers are permanently as well as temporarily cationic, anionic, non-ionic or amphoteric polymers. The film-forming polymers may be of synthetic or natural origin.

Examples of common polymers are acrylamide/ammonium acrylate copolymer, acrylamides/DMAPA acrylates/methoxy PEG methacrylate copolymer, acrylamidopropyltrimonium chloride/acrylamide copolymer, acrylamidopropyltrimonium chloride/acrylates copolymer, acrylates/acetoacetoxyethyl methacrylate copolymer, acrylates/acrylamide copolymer, acrylates/ammonium methacrylate copolymer, acrylates/t-butyl acrylamide copolymer, acrylates copolymer, acrylates/C1-2 succinates/hydroxyacrylates copolymer, acrylates/lauryl acrylate/stearyl acrylate/ethylamine oxide methacrylate copolymer, acrylates/octyl acrylamide copolymer, acrylates/octyl acrylamide/diphenyl amodimethicone copolymer, acrylates/stearyl acrylate/ethylamine oxide methacrylate copolymer, acrylates/VA copolymer, acrylates/VP copolymer, adipic acid/diethylenetriamine copolymer, adipic acid/dimethylaminohydroxypropyl diethylenetriamine copolymer, adipic acid/epoxypropyl diethylenetriamine copolymer, adipic acid/isophthalic acid/neopentylglycol/trimethylolpropane copolymer, allyl stearate/VA copolymer, aminoethylacrylate phosphate/acrylates copolymer, aminoethylpropanediol-acrylates/acrylamide copolymer, aminoethylpropanediol-AMPD-acrylates/diacetone-acrylamide copolymer, ammonium VA/acrylates copolymer, AMPD-acrylates/diacetone-acrylamide copolymer, AMP-acrylates/allyl methacrylate copolymer, AMP-acrylates/C1-18 alkyl acrylates/C1-8 alkyl acrylamide copolymer, AMP-acrylates/diacetoneacrylamide copolymer, AMP-acrylates/dimethylaminoethyl methacrylate copolymer, bacillus/rice bran extract/soybean extract ferment filtrate, bis-butyloxyamodimethicone/PEG-60 copolymer, butyl acrylate/ethylhexyl methacrylate copolymer, butyl acrylate/hydroxypropyldimethicone acrylate copolymer, butylated PVP, butyl ester of ethylene/MA copolymer, butyl ester of PVM/MA copolymer, calcium/sodium PVM/MA copolymer, corn starch/acrylamide/sodium acrylate copolymer, diethyleneglycolamine/epichlorohydrin/piperazine copolymer, dimethicone crosspolymer, diphenyl amodimethicone, ethyl ester of PVM/MA copolymer, hydrolyzed wheat protein/PVP crosspolymer, isobutylene/ethylmaleimide/hydroxy-ethylmaleimide copolymer, isobutylene/MA copolymer, isobutyl methacrylate/bis-hydroxypropyl dimethicone acrylate copolymer, isopropyl ester of PVM/MA copolymer, lauryl acrylate crosspolymer, lauryl methacrylate/glycol dimethacrylate crosspolymer, MEA-sulfite, methacrylic acid/sodium acrylamide-methyl-propane sulfonate copolymer, methacryloyl ethyl betaine/acrylates copolymer, octyl acrylamide/acrylates/butylaminoethyl methacrylate copolymer, PEG/PPG-25/25 dimethicone/acrylates copolymer, PEG-8/SMDI copolymer, polyacrylamide, polyacrylate-6, polybeta-alanine/glutaric acid crosspolymer, polybutylene terephthalate, polyester-1, polyethylacrylate, polyethylene terephthalate, polymethacryloyl ethyl betaine, polypentaerythrityl terephthalate, polyperfluoroperhydrophenanthrene, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-39, polyquaternium-45, polyquaternium-46, polyquaternium-47, polyquaternium-48, polyquaternium-49, polyquaternium-50, polyquaternium-55, polyquaternium-56, polysilicone-9, polyurethane-1, polyurethane-6, polyurethane-10, polyvinyl acetate, polyvinyl butyral, polyvinylcaprolactam, polyvinylformamide, polyvinyl imidazolinium acetate, polyvinyl methyl ether, potassium butyl ester of PVM/MA copolymer, potassium ethyl ester of PVM/MA copolymer, PPG-70 polyglyceryl-10 ether, PPG-12/SMDI copolymer, PPG-51/SMDI copolymer, PPG-10 sorbitol, PVM/MA copolymer, PVP, PVP/VA/itaconic acid copolymer, PVP/VA/vinyl propionate copolymer, rhizobian gum, rosin acrylate, shellac, sodium butyl ester of PVM/MA copolymer, sodium ethyl ester of PVM/MA copolymer, sodium polyacrylate, sterculia urens gum, terephthalic acid/isophthalic acid/sodium isophthalic acid sulfonate/glycol copolymer, trimethylolpropane triacrylate, trimethylsiloxysilylcarbamoyl pullulan, VA/crotonates copolymer, VA/crotonates/methacryloxybenzophenone-1 copolymer, VA/crotonates/vinyl neodecanoate copolymer, VA/crotonates/vinyl propionate copolymer, VA/DBM copolymer, VA/vinyl butyl benzoate/crotonates copolymer, vinylamine/vinyl alcohol copolymer, vinyl caprolactam/VP/dimethylaminoethyl methacrylate copolymer, VP/acrylates/lauryl methacrylate copolymer, VP/dimethylaminoethyl methacrylate copolymer, VP/DMAPA acrylates copolymer, VP/hexadecene copolymer, VP/VA copolymer, VP/vinyl caprolactam/DMAPA acrylates copolymer, yeast palmitate and styrene/VP copolymer.

With particular preference the polymer is selected from the group of non-ionic polymers. Suitable non-ionic polymers are for example:
Polyvinyl pyrrolidones, such as those sold under the name Luviskol® (BASF).
Vinyl pyrrolidone/vinyl ester copolymers, such as those sold under the trademark Luviskol® (BASF). Luviskol® VA 64 and Luviskol® VA 73, both vinyl pyrrolidone/vinyl acetate copolymers, are preferred non-ionic polymers.
Cellulose ethers, such as hydroxypropyl cellulose, hydroxyethyl cellulose and hydroxypropyl methyl cellulose, as they are marketed under the trademarks Culminal® and Benecel® (AQUALON).
Shellac.
Siloxanes. These siloxanes may be both water-soluble and water-insoluble. Both volatile and non-volatile siloxanes are suitable. Non-volatile siloxanes are compounds whose boiling point at normal pressure is above about 200° C. Preferred siloxanes are polydialkyl siloxanes such as polydimethyl siloxane, polyalkyl aryl siloxanes such as polyphenyl methyl siloxane, ethoxylated polydialkyl siloxanes and polydialkyl siloxanes containing amine and/or hydroxy groups.

As contemplated herein, polyvinyl pyrrolidones and vinyl pyrrolidone/vinyl acetate copolymers are preferred in particular.

Amphoteric polymers form a second group of particularly preferred polymers. The use of a copolymer of i) N-tert-octylacrylamide, ii) acrylic acid, iii) tert-butylaminoethyl methacrylate and optionally other monomers is very particularly preferred.

Preferred amphoteric copolymers preferably include at least about 90% by weight, preferably at least about 95% by weight and in particular at least about 97% by weight of the monomers N-tert-octylacrylamide, acrylic acid and tert-butylaminoethyl methacrylate. Corresponding copolymers may also be obtained using exclusively the monomers N-tert-octylacrylamide, acrylic acid and tert-butylaminoethyl methacrylate.

Also preferred are copolymers of the monomers N-tert-octylacrylamide, acrylic acid, tert-butylaminoethyl methacrylate, methyl methacrylate and hydroxypropyl methacrylate. It is particularly preferred if the copolymer includes at least about 90% by weight, preferably at least about 95% by weight and in particular at least about 97% by weight of the monomers N-tert-octylacrylamide, acrylic acid, tert-butylaminoethyl methacrylate, methyl methacrylate and hydroxypropyl methacrylate.

The amphoteric copolymers described above are marketed, for example, under the name Amphomer® (INCI designation: octylacrylamide/acrylates/butylaminoethyl methacrylate copolymer; CAS number 70801-07-9) by National Starch.

A fourth essential component of the cosmetic compositions is the propellant. It has proved to be of particular cosmetic and technical advantage if the weight percentage of the propellant in the total weight of the composition is from about 25 to about 50% by weight, preferably from about 30 to about 50% by weight and in particular from about 35 to about 45% by weight.

Preferred propellants (propellant gases) are selected from propane, propene, n-butane, isobutane, isobutene, n-pentane, pentene, isopentane, isopentene, methane, ethane, dimethyl ether, nitrogen, air, oxygen, nitrous oxide, dichlorofluoromethane, chlorodifluoromethane, chlorofluoromethane, 1,1,2,2-tetrachloro-1-fluoroethane, 1,1,1,2-tetrachloro-2-fluoroethane, 1,2,2-trichloro-1,1-difluoroethane, 1,1,2-trichloro-1,2-difluoroethane, 1,1,1-trichloro-2,2-difluoroethane, 2,2-dichloro-1,1,1-trifluoroethane, 1,2-dichloro-1,1,2-trifluoroethane, 2-chloro-1,1,1,2-tetrafluoroethane, 1-chloro-1,1,2,2-tetrafluoroethane, 1,1,2-trichloro-2-fluoroethane, 1,2-dichloro-1,2-difluoroethane, 1,2-dichloro-1,1-difluoroethane, 1-chloro-1,2,2-trifluoroethane, 2-chloro-1,1,1-trifluoroethane, 1-chloro-1,1,2-trifluoroethane, 1,2-dichloro-1-fluoroethane, 1,1-dichloro-1-fluoroethane, 2-chloro-1,1-difluoroethane, 1-chloro-1,1-difluoroethane, 1-chloro-2-fluoroethane, 1-chloro-1-fluoroethane, 2-chloro-1,1-difluoroethene, 1,1,1,3-tetrafluoroethane, heptafluoro-n-propane, perfluoroethane, monochlorodifluoromethane, 1,1-difluoroethane, both individually and in combination.

Preferred is the use of propane, propane/butane mixtures or dimethylether, especially preferred is the use of dimethylether.

As a fifth and/or sixth component, the cosmetic compositions as contemplated herein contain at least one coloring compound from the group of dyes and/or pigments. The cosmetic compositions as contemplated herein contain
from 0 to about 10% by weight of dye(s) (e) and/or,
from 0 to about 10% by weight of pigment(s) (f),
   with the proviso that the sum of the amounts of ingredients (e) and (f) is from about 0.0001 to about 10% by weight. In other words, the compositions as contemplated herein may contain 0% by weight of dyes (then at least one pigment must be present in an amount of at least about 0.0001% by weight), but the dye content may also be about 10% by weight (then no pigment may be present.) Irrespective of whether
only one dye,
several dyes,
only one pigment,
several pigments,
one dye and several pigments,
several dyes and one pigment,
several dyes and several pigments
   are used, the total amount of all dyes and pigments (also referred to as total amount of coloring components) on the average as contemplated herein is from about 0.0001 to about 10% by weight.

Preferred cosmetic compositions as contemplated herein contain from about 0.0001 to about 10% by weight, preferably from about 0.01 to about 9.5% by weight, more preferably from about 0.05 to about 9% by weight, still more preferably from about 0.1 to about 8.5% by weight and in particular from about 0.25 to about 8% by weight of dye(s).

Dyes are colorants, i.e. coloring substances which, unlike pigments, are soluble in the application medium (as in water, oils or other solvents).

Depending on the desired color, color intensity and fastness properties of the resulting coloration, a dye or a mixture of dyes may be selected. All dyes commonly used and commercially available in the field of cosmetics may be used without any problems within the scope of the present disclosure.

Likewise, preferred cosmetic compositions in accordance with the present disclosure contain from about 0.0001 to about 10% by weight, preferably from about 0.01 to about 9.5% by weight, more preferably from about 0.05 to about 9% by weight, still more preferably from about 0.1 to about 8.5% by weight and in particular from about 0.25 to about 8% by weight of pigment(s).

In principle, all types of water-insoluble pigments are suitable, for example natural inorganic pigments (also known as mineral pigments). These pigments mainly contain sulfides and oxides. Examples of such pigments are ochre (Fe(OOH); pigment yellow 43), burnt sienna (Fe₂O₃; pigment red 102), umbra (Fe₂O₃ x MnO₂; pigment brown 7:x), cinnabar (β-HgS, PR 106), lapis lazuli (ultramarine, Na₆Al₆Si₆O₂₄ x Na₂Sn; pigment blue 29), azurite (basic copper carbonate, Cu₃[OH/CO₃]₂; PB 30), green earth (FeO-containing silicate; pigment green 23), malachite (Cu₂[OH)₂, CO₃]) and carbon black (carbon (graphite), pigment black 9). However, the use of synthetic inorganic pigments has proven to be advantageous with regard to the avoidance of undesirable visible residues or grey haze or the water-resistant temporary coloring of the fibers. Synthetic inorganic pigments are produced, for example, by chemical and/or physical conversion (digestion, precipitation, annealing). These include in particular
white pigments (titanium dioxide (TiO₂), pigment white PW 6; zinc sulphide (ZnS), PW 7; zinc oxide (ZnO), PW 4; antimony white (Sb₂O₃), PW 11; lithopones (ZnS/BaSO₄), PW 5; lead white (2PbCO₃ x Pb(OH)₂), PW 1),
secondary white fillers (calcium carbonate, PW 18; talcum, PW 26 and barium sulphate, PW 21),
black pigments (manganese black, spinel black and pigment blacks (graphite-carbon),
luster pigments (absorption pigments, metallic pigments or metallic effect pigments and pearlescent pigments), and
inorganic colored pigments (iron oxide pigments, iron blue pigments, ultramarine pigments as well as lead chromate pigments, chromium oxide pigments, cadmium pigments and bismuth vanadate pigments, which are less suitable due to their toxicological properties).

Preferred synthetic inorganic pigments are metal pigments or metal effect pigments made of powdery metals or metal alloys, such as aluminum bronzes (metal: Al), gold bronzes (metal: Cu, Cu—Al or Cu—Zn alloy), silver bronzes (metal: Cu—Zn—Ni), fire-colored bronzes (metal: oxidized Cu—Zn) and patent bronzes (metal: Cu—Zn—(Ni)+dye).

Other preferred synthetic inorganic pigments are pearlescent pigments, which include several layers with different refractive indices. Examples of such pearlescent pigments are magnesium stearate, zinc stearate and lithium stearate or ethylene glycol distearate or polyethylene terephthalate, as well as pearlescent pigments which primarily include mica, titanium dioxide (titanium dioxide mica), bismuth chloride oxide or guanine, and may also be coated with colored oxide layers (e.g. iron oxides or chromium oxides). Pearlescent pigments based on mica and mica/metal oxide are particularly preferred pearlescent pigments. Mica belongs to the group of layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide. Suitable metal oxides include TiO₂, Cr₂O₃ and Fe₂O₃. By appropriate coating, interference pigments as well as color luster pigments are obtained as pearlescent pigments preferred by the present disclosure. These types of pearlescent pigments have a glittering optical effect as well as color effects. Furthermore, the pearlescent pigments that may be used as contemplated herein may additionally contain a color pigment that is not derived from a metal oxide.

Particularly preferred pearlescent pigments are those marketed by Merck under the trade name Colorona®, whereby the pigments Colorona® red-brown (from about 47-57% by weight muscovite mica (KH₂(AlSiO₄)₃), from about 43-50% by weight Fe₂O₃ (INCI: iron oxides CI 77491), <3% by weight TiO₂ (INCI: titanium dioxide CI 77891), Colorona® Blackstar Blue (from about 39-47% by weight muscovit mica (KH₂(AlSiO₄)₃), from about 53-61% by weight Fe₃O₄ (INCI: iron oxides CI 77499)), Colorona® Siena Fine (from about 35-45% by weight muscovit mica (KH₂(AlSiO₄)₃), from about 55-65% by weight Fe₂O₃ (INCI: iron oxides CI 77491)), Colorona® Aborigine Amber (from about 50-62% by weight muscovit mica (KH₂(AlSiO₄)₃), from about 36-44% by weight Fe₃O₄ (INCI: iron oxides CI 77499), from about 2-6% by weight TiO₂ (INCI: titanium dioxide CI 77891)), Colorona® Patagonian Purple (from about 42-54% by weight muscovit mica (KH₂(AlSiO₄)₃), from about 26-32% by weight Fe₂O₃ (INCI: iron oxides CI 77491), from about 18-22% by weight TiO₂ (INCI: titanium dioxide CI 77891), from about 2-4% by weight Prussian blue (INCI: ferric ferrocyanide CI 77510)), Colorona® Chameleon (from about 40-50% by weight muscovit mica (KH₂(AlSiO₄)₃), from about 50-60% by weight Fe₂O₃ (INCI: iron oxides CI 77491)) and Silk® mica (>98% by weight muscovit mica (KH₂(AlSiO₄)₃)) are particularly preferred.

A group of particularly preferred pigments are the coloring synthetic iron oxides. Particularly preferred representatives of this substance class are pigment brown 6 (CI no. 77491), pigment red 101 (CI no. 77491), pigment yellow 42 (CI no. 77492), pigment black 11 (CI no. 77499) and mixtures of these pigments.

Particularly preferred pigments that may be used as contemplated herein are exemplified by good sprayability in the compositions as contemplated herein, a homogeneous spray pattern and an extremely low tendency to clog the spray nozzles. Extremely preferred cosmetic compositions are exemplified by at least one pigment from the group CI12490, CI14700, CI14720, CI15510, CI15985, CI45380, CI47005, CI60730, CI61565, CI73360, CI74160, CI77007, CI77019, CI77288, CI77289, CI77491, CI77492, the total amount of pigment(s) from this group being 0.0001 to 10% by weight, preferably from about 0.01 to about 9.5% by weight, more preferably from about 0.05 to about 9% by weight, still more preferably from about 0.1 to about 8.5% by weight and particularly preferably from about 0.25 to about 8% by weight.

With particular preference, the products as contemplated herein contain at least one starch compound. This starch compound may be the only polymer in the agents as contemplated herein, but it is preferred to use the starch compound in addition to the preferably film-forming polymers described above. The phrase “as an additional ingredient” therefore means “in addition to” all other mandatory ingredients mentioned above.

Preferred cosmetic compositions as contemplated herein contain as a further ingredient from about 1.0 to about 10% by weight, preferably from about 2.0 to about 6.0% by weight and in particular from about 3.0 to about 5.0% by weight of at least one starch compound.

Starch is a reserve carbohydrate that is stored by many plants in the form of starch grains (granules), usually between about 1 and about 200 μm large, in various parts of the plant, e.g. in tubers or roots, cereal seeds, fruits as well as in the pith. Starch belongs to the family of homoglycans and is a polycondensation product of D-glucose. Starches preferred as contemplated herein are selected from at least one polycondensation product of D-glucose obtained from native and/or physically modified starch of potatoes, corn, rice, peas, acorns, chestnuts, barley, wheat, bananas, sago, millet, sorghum, oats, rye, beans, batata, maranta or manioc as well as mixtures thereof. In terms of cleaning performance, the use of native and/or physically modified rice starch has proven to be particularly advantageous. Native starch is a starch which is isolated from starch-containing plants and which has not been physically or chemically modified after isolation and purification. Physically modified starch, on the other hand, is starch which has been subjected to at least one physical modification after isolation. Physical modification in this context means modification by application of pressure and/or heat and/or light. A modification by employing chemical and enzymatic reactions, for example the hydrolysis of starch, does not fall under the term physical modification. A preferred physical modification is the application of heat, especially cooking of the native starch. Preferred embodiments of the present disclosure are exemplified by compositions contain at least one starch selected from chemically and/or physically modified rice starches, in particular physically modified rice starch. By using native and/or physically modified rice starches in combination with dyes and/or pigments, a particularly high adhesion of the dyes and/or pigments to the starch is achieved, so that, in addition to the excellent cleaning performance, long-lasting temporary hair coloration is also made possible.

As a result, it is possible and preferred to use pre-colored and/or pre-pigmented starches which can be sprayed outstandingly with the compositions as contemplated herein. Particularly preferred cosmetic compositions contain the ingredient (e) in the form of a starch-dyeing compound, preferably in the form of dyed and/or pigmented rice starch.

In addition, it has been found that the composition of the starch particles themselves has been shown to be relevant for the cosmetic effect. Therefore, preferably used particles contain a certain proportion of native and/or physically modified starch, especially rice starch. It is thus preferred as contemplated herein if the starch-dye compound contains at least one starch, in particular a physically modified rice starch, in a total amount of from about 70 to about 96% by weight, in particular from about 80 to about 94% by weight, in each case based on the total weight of the particle (compound). The use of particles which contain a high proportion by weight of physically modified rice starch results in particularly homogeneous spray patterns and preferential adhesion of the color to the keratinous fibers.

In addition to such compounds of starch and coloring substances or in their place, the fibers of the compositions as contemplated herein may naturally also be pre-colored and/or pre-pigmented. Here, cosmetic compositions as contemplated herein are particularly preferred, which contain the ingredients (a) and (e) or (f) in the form of a fiber-dyeing compound, preferably in the form of colored and/or pigmented fibers.

Preferred cosmetic compositions contain at least one ester oil as a further component. The group of ester oils includes in particular esters of C6-C30 fatty acids with C2-C30 fatty alcohols, preferably monoesters of the fatty acids with alcohols containing 2 to 24 C atoms. Examples of ester oils are isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyloleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), Di-n-butyl adipate (Cetiol® B), myristyl myristate (Cetiol® MM), cetearyl isononanoates (Cetiol® SN) and decyl oleate (Cetiol® V). The use of isopropyl myristate is particularly preferred.

The percentage by weight of ester oil in the total weight of the cosmetic preparation is preferably from about 0.01 to about 1.0% by weight, preferably from about 0.02 to about 0.6% by weight and in particular from about 0.03 to about 0.2% by weight.

The fibers described at the beginning as the first essential component of the cosmetic compositions may be modified in various ways. Such modifications concern for example the length of the fibers described above or their length-to-diameter ratio.

Another option to influence the technical and cosmetic properties of the compositions is to modify the surface properties of the fibers used.

In a first preferred embodiment hydrophobic fibers are used. The hydrophobicity (impregnation) causes a water-repellent finish of the fiber. Suitable water-repellent agents are paraffins or waxes, but also film-forming silicones.

Depending on the intended use of the cosmetic composition it may be advantageous if the fibers have a certain oil absorption capacity. This oil absorption capacity may be influenced by the hydrophobicity of the fibers described above. The use of hydrophobic or non-hydrophobic fibers with an oil absorption capacity above about 0.2 g jojoba oil per gram fiber is preferred. Particularly preferred is the use of fibers with an oil absorption capacity (at about 20° C.) of from about 0.2 to about 0.8 g jojoba oil/g, preferably of from about 0.3 to about 0.7 g jojoba oil/g, particularly preferably of from about 0.4 to about 0.6 g jojoba oil/g.

For example, in order to allow a uniform suspension of the fibers in the cosmetic composition or to increase the cosmetic effect of the composition, the compositions may include surfactant as a further optional ingredient. The use of non-ionic or cationic surfactants is preferred.

Preferred cationic surfactants are selected from the group of quaternary ammonium compounds. Preferred quaternary ammonium compounds are again amonium halides, in particular chlorides and bromides, such as alkyl trimethyl ammonium chlorides, dialkyldimethyl ammonium chlorides and trialkyl methyl ammonium chlorides, e.g. cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride, as well as the imidazolium compounds known under the INCI designations quaternium 27 and quaternium 83. The use of cetyl trimethyl ammonium chloride is particularly preferred.

The percentage by weight of the surfactant in the total weight of the cosmetic composition is preferably from about 0.05 to about 2.0% by weight, preferably from about 0.1 to about 1.5% by weight and more particularly from about 0.2 to about 1.0% by weight.

In an alternative embodiment, hydrophilized fibers are used in the cosmetic compositions. Compared with the non-hydrophilized fibers, corresponding fibers have an increased water absorption capacity.

The water adsorption capacity (at about 20° C.) of preferred fibers, in particular preferred hydrophilized fibers, is preferably above about 5.0% by weight, preferably above about 7.0% by weight and in particular above about 9.0% by weight of the net weight of the fibers.

The hydrophilization of the fibers improves, among other things, their water absorption capacity. Corresponding fibers are therefore used especially in refreshing cosmetic compositions.

In another alternative embodiment, preferred cosmetic compositions contain at least one fragrance.

Optional further suitable active substances or auxiliaries which may be contained in the cosmetic compositions are in particular additional care substances.

As a care substance, the composition may for example contain at least one protein hydrolysate and/or one of its derivatives. Protein hydrolysates are product mixtures obtained by acid, basic or enzymatic catalyzed degradation of proteins. As contemplated herein, the term protein hydrolysates also includes total hydrolysates as well as individual amino acids and their derivatives and mixtures of different amino acids. The molecular weight of the protein hydrolysates which may be used as contemplated herein is between about 75, the molecular weight for glycine, and about 200,000, preferably the molecular weight is from about 75 to about 50,000 and particularly preferably from about 75 to about 20,000 daltons.

As a skin care substance, the agent as contemplated herein may furthermore contain at least one vitamin, one provitamin, one vitamin precursor and/or one of their derivatives. As contemplated herein, such vitamins, provitamins and vitamin precursors are preferred which are usually assigned to the groups A, B, C, E, F and H.

Further care substances are panthenol, caffeine, nicotinamide and sorbitol.

As a care substance, the products as contemplated herein may furthermore contain at least one plant extract, but also mono- or oligosaccharides and/or lipids.

Aerosol dispensing containers are particularly suitable for dispensing and applying the cosmetic compositions. A further subject-matter of the application are cosmetic compositions comprising
a) a cosmetic composition as contemplated herein, and
b) an aerosol dispenser.

The cosmetic products covered by the present disclosure may be manufactured in the usual manner As a general rule, all the ingredients of the cosmetic composition, except the propellant, shall be placed in a suitable pressure-resistant container. This container is then closed with a valve. Finally, the desired quantity of the propellant is filled in using conventional techniques.

Pressure-resistant containers may be made of metal (aluminum, tinplate, tin), protected or non-splintering plastic or made of glass, which is coated on the outside with plastic. The selection of these containers must take into account resistance to pressure and breakage, corrosion resistance, easy filling, aesthetic aspects, ease of handling, printability, etc. Special internal protective coatings guarantee corrosion resistance to the composition packaged in the pressure vessel. Particularly preferred are valves with an internally painted valve head, whereby the paint and valve material are compatible with each other. If aluminum valves are used, their valve heads may be coated on the inside, e.g. with micoflex lacquer. If tinplate valves are used as contemplated herein, their valve heads may be coated on the inside with PET (polyethylene terephthalate), for example.

A multi-chamber dispenser may also be used as an aerosol dispensing container. The multi-chamber dispenser may also be designed in such a way that one chamber contains the compressed propellant and another chamber is filled with the remaining components of the composition. Such a multi-chamber dispenser is for example a so-called bag-in-can packaging.

The spray rate of the cosmetic products is preferably from about 6.5 to about 10.0 g/10 s.

As mentioned above, the products as contemplated herein are particularly suitable for cleaning, refreshing and deodorizing keratinous fibers. Corresponding uses of the cosmetic compositions are further subject-matters of this application.

A final subject-matter of this application is a process for the temporary shaping of keratinous fibers, in particular human hair, in which a cosmetic composition as contemplated herein is applied to keratinous fibers. The ingredients of that composition, their percentages by weight and preferred embodiments are subject, mutatis mutandis, to the provisions set out above.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for the cosmetic treatment of keratinous fibers, comprising, in each case based on a total weight of the cosmetic composition:
(a) fibers,
(b) ethanol,
(c) at least one polymer,
(d) a propellant,
(e) from 0 to about 10% by weight of a dye,
(f) from 0 to about 10% by weight of a pigment,
wherein a sum of the quantities of ingredients (e) and (f) is from about 0.0001 to about 10% by weight.

2. The cosmetic composition according to claim 1, wherein the percentage by weight of the fibers in the total weight of the cosmetic composition is from about 0.1 to about 2.0% by weight.

3. The cosmetic composition according to claim 1, wherein the cosmetic composition comprises from about 0.0001 to about 10% by weight of the dye, based on the total weight of the cosmetic composition.

4. The cosmetic composition according to claim 1, wherein the cosmetic composition comprises from about 0.0001 to about 10% by weight of the pigment, based on the total weight of the cosmetic composition.

5. The cosmetic composition according to claim 1, wherein the cosmetic composition comprises at least one pigment selected from the group of CI12490, CI14700, CI14720, CI15510, CI15985, CI45380, CI47005, CI60730, CI61565, CI73360, CI74160, CI77007, CI77019, CI77288, CI77289, CI77491, CI77492, wherein the total amount of pigment from this group is from about 0.0001 to about 10% by weight, based on a total weight of the cosmetic composition.

6. The cosmetic composition according to claim 1, wherein the fibers have a length of from about 1.0 to about 200 μm.

7. The cosmetic composition according to claim 1, wherein the cosmetic composition comprises from about 0.5 to about 8.0% by weight of the at least one polymer, base on total weight of the cosmetic composition.

8. The cosmetic composition according to claim 1, wherein the cosmetic composition comprises from about 20 to about 50% by weight of the ethanol, based on the total weight of the cosmetic composition.

9. The cosmetic composition according to claim 1, wherein the cosmetic composition comprises from about 30 to about 70% by weight of the propellant, based on the total weight of the cosmetic composition.

10. The cosmetic composition according to claim 1, wherein the cosmetic composition comprises as a further component from about 1.0 to about 10% by weight of at least one starch compound, based on the total weight of the cosmetic composition.

11. The cosmetic composition according to claim 1, wherein the cosmetic composition comprises the ingredient (e) in the form of a starch-dye compound.

12. The cosmetic composition according to claim 1, wherein the cosmetic composition comprises the ingredients (a) and (e) in the form of a fiber-dye compound.

13. The cosmetic composition of claim 1, wherein the cosmetic composition comprises the pigment in the form of a fiber-pigment compound.

14. The cosmetic composition of claim 1, wherein the cosmetic composition comprises the dye in the form of a dyed rice starch.

15. The cosmetic composition of claim 1, wherein the cosmetic composition comprises the pigment in the form of a pigmented rice starch.

16. The cosmetic composition of claim 1, wherein the cosmetic composition comprises the dye in the form of a dyed fiber.

17. The cosmetic composition of claim 1, wherein the cosmetic composition comprises the pigment in the form of a pigmented fiber.

18. The cosmetic composition of claim 1, wherein the cosmetic composition comprises fibers in an amount of from about 0.1 to about 0.5 weight percent, based on the total weight of the cosmetic composition.

19. The cosmetic composition of claim 1, wherein the cosmetic composition comprises the dye in an amount of from about 0.25 to about 8 weight percent, based on the total weight of the cosmetic composition.

20. The cosmetic composition of claim 1, wherein the cosmetic composition comprises the pigment in an amount of from about 0.25 to about 8 weight percent, based on the total weight of the cosmetic composition.
